(19) United States
(12) Patent Application Publication
(10) Pub. No.: US 2023/0357709 A1
(43) Pub. Date: Nov. 9, 2023

(54) 3D STRUCTURES FOR CELL GROWTH

(71) Applicants: UNIVERSITEIT MAASTRICHT, MAASTRICHT (NL); ACADEMISCH ZIEKENHUIS MAASTRICHT, MAASTRICHT (NL); NEDERLANDSE ORGANISATIE VOOR WETENSCHAPPELIJK ONDERZOEK (NWO), CE DEN HAAG (NL)

(72) Inventors: Paul Andrew WIERINGA, MAASTRICHT (NL); Lorenzo MORONI, MAASTRICHT (NL); Victor Retamero DE LA ROSA, GENT (BE); Richard HOOGENBOOM, TERNEUZEN (NL)

(21) Appl. No.: 18/029,391

(22) PCT Filed: Sep. 30, 2021
(86) PCT No.: PCT/EP2021/076925

§ 371 Date: Mar. 30, 2023

(30) Foreign Application Priority Data
Sep. 30, 2020 (EP) 20199196.5

(51) Int. Cl. C12N 5/00; B33Y 80/00; C08J 3/075; C08J 3/24; B33Y 40/20; B33Y 70/00; C12M 1/12; B29C 64/30; B29C 64/118; C08G 73/22

(52) U.S. Cl. CPC C12N 5/0062; B29C 64/118; B29C 64/30; B33Y 40/20; B33Y 70/00; B33Y 80/00; C08G 73/22; C08J 3/075; C08J 3/24; C12M 25/14; B29K 2079/00; C12N 2513/00; C12N 2533/30; C12N 2533/54

(57) ABSTRACT

The present invention relates to a method for creating a three- dimensional structure for cell growth by creating a template of a polymer, applying a hydrogel support onto the template and removing the template. The present invention further provides a device for cell growth comprising a polymer template embedded in a hydrogel.

The present invention relates to a method for creating a three-dimensional structure for cell growth by creating a template of a polymer, applying a hydrogel support onto the template and removing the template. The present invention further provides a device for cell growth comprising a polymer template embedded in a hydrogel.

### BACKGROUND

It has been a major focus of tissue engineering to create increasingly more complex and representative 3D tissue constructs, either as more relevant in vitro platforms to study biological processes or to develop more competent replacement tissues. A critical challenge in achieving the goal has been the creation of lumen structures, a prevalent feature throughout the body including the intestine, blood vessels, bile duct, fallopian tubes, loops of Henle, etc. Whether they transport elements from one location to another or provide structural guidance for cell function, growth, and repair, the generation of a biologically relevant environment that incorporates appropriately sized lumen with meaningful organization has been an intense area of research in the translation from 2D cell biology to 3D tissue systems.

In view of this, there is research ongoing to create oriented arrays of microchannels that would be capable of supporting and directing cell growth. Prior art options for creating microchannels include “gel-in-gel” constructions, which are large and bulky and must rely on cell organisation, and fugitive inks which are typically mechanically weak constructions.

Other research is directed towards the use of sacrificial templates that form the desired structures embedded in a hydrogel. For instance, sugar is used, which has the drawbacks that it is soluble and must be coated, that it cannot easily be pre-seeded, that sugar is cytotoxic and that the template cannot be as small as desired.

One of the mechanisms for creating a sacrificial template is the use of polymers that have a thermally induced solubility. These polymers have a so-called Lower Critical Solubility Temperature (LCST). Above this temperature, the polymers are hydrophobic and retain their mechanical integrity allowing the structure to be embedded within a hydrogel. After cooling to a temperature below the LCST, the polymer will transition to its hydrophilic state, allowing it to become water soluble.

One known polymer that is used is pNIPAM (Poly(N-isopropylacrylamide)). With this polymer, some desirable results are obtained, but it still has some drawbacks in that the temperature transition range is relatively narrow and that it is too hydrophilic to be pre-seeded with cells.

For example, Haigh et al. (Macromol. Rapid Commun. 2016, 37, 93-99) disclose a method using 3D printing of a sacrificial template composed of poly(E-caprolactone) inside a hydrogel, however a drawback of the method is that it does not allow seeding of living cells on the sacrificial template prior to embdedding.

Thus, in order for a polymer to be suitable to create such a sacrificial template, it should be able to be processed into relatively small filaments by means of additive manufacturing or electrospinning techniques. It should also retain its mechanical integrity above the LCST while being embedded in the aqueous pre-polymer hydrogel solution. It should further be possible to remove the polymer relatively easily below the LCST by dissolving it into biocompatible, preferably aqueous medium. And preferably, the polymer should allow pre-seeding with cells.

It is thus an object of the invention to provide a method for creating a three-dimensional structure for cell growth, by using a sacrificial template that fulfils the requirements as described above.

### INVENTION

The present invention provides a method for creating three-dimensional structures for cell growth comprising the steps of:
a) providing a polymer;
b) creating a template from the polymer;
c) applying a hydrogel support onto the template to embed the template in the hydrogel;
   wherein the polymer is a PsecBuOx-stat-PAOx co-polymer represented by formula (I); 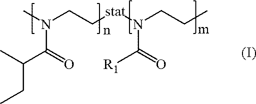
   wherein each R₁ is independently selected from -methyl, -ethyl, -n-propyl, cyclo-propyl and iso-propyl;
   the sum of m and n is from 20 to 1000, preferably from 200 to 700, most preferably from 300 to 500. Optionally the method further comprises the step of:
d) removing the template.

An advantage of the invention is that the relatively benign trigger for template dissolution, i.e. lowering the temperature in an aqueous medium, allows the hydrogel to retain its original mechanical properties, and furthermore, biologicals (proteins, growth factors, cells) can be safely incorporated within the structure while retaining their viability throughout the dissolution process.

### Polyoxazolines

A PsecBuOx-stat-PAOx co-polymer is also referred to as a Polyoxazoline. Polyoxazolines are known as carriers for drug delivery. Poly(2-oxazoline)s or poly(2-alkyl/aryl-2-oxazolines) (commonly abbreviated as PAOs, Pox, or POZ) are readily obtained via the cationic ring-opening polymerization of 2-oxazolines. They are for instance described in WO2013103297 and WO2019175434.

The thermoresponsive properties of polyoxazoline derivatives are known in the art, and their use as materials for controlled cell-adhesion has been proposed. However, the application of polyoxazolines is to date limited to films on bidimensional surfaces due to the poor mechanical properties of these materials. Recent attempts to overcome these issues by copolymerization have been unsuccessful. Indeed, as observed in other polymers discussed, when thermoresponsive polyoxazoline mats and molds were submerged in water at a temperature above the cloud point temperature (T_{CP} - when used herein T_{CP} is the cloud point transition temperature at a specific polymer concentration), they remained undissolved but lost their shape stability. See for instance in: Oleszko-Torbus, N., et al. “Poly(2-oxazoline) Matrices with Temperature-Dependent Solubility-Interactions with Water and Use for Cell Culture”; Materials 2020, 13, 2702.

Polyoxazoline) polymers can be described by the general formula II:

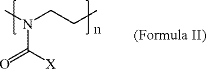

wherein X is a linear or branched C1-C5 alkyl or a cyclopropyl.

Depending on the nature of X, the polymer will show a different hydrophobicity and LCST. Representative polyoxazolines are shown in Table 1.

TABLE 1

| | | |
|---|---|---|
| X | Abbreviation | LCST |
| methyl | PMeOx | No LCST |
| ethyl | PEtOx | ~60° C. |
| iso-propyl | PiPrOx | ~35° C. |
| cyclopropyl | PcPrOx | ~30° C. |
| n-propyl | PnPrOx | ~25° C. |
| sec-Butyl | PsecBuOx | ~0-5° C. |
| n-Butyl | PnButOx | Water insoluble |

The lower critical solution temperature (LCST) should be understood as a temperature at which a material acquires or loses a distinctive property. In this case, it specifically concerns the transition between hydrophobic and hydrophilic properties of a material upon cooling. For example, when a co-polymer reaches or exceeds its transition temperature, the material acquires hydrophobic properties (during heating) or hydrophilic properties (during cooling).

The polyoxazoline used in the method of the present invention comprises a PsecBuOx-PAOx backbone; wherein PAOx represents poly(2-alkyl-2-oxazoline), and alkyl may be selected from any one of methyl, ethyl, propyl and its isoforms (n-propyl, iso-propyl and cyclo-propyl), or combinations thereof.

The polyoxazoline can be described with the above Formula I

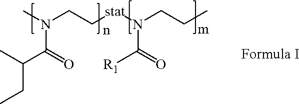

Formula I thus describes a compound of Formula II wherein X is methyl, ethyl, propyl or sec-butyl. The compound of Formula I can also be described as a copolymer of PAOx (PMeOx, PEtOx, PPrOx) and PsecBuOx having different molar ratios of PAOx and PsecBuOx. PAOx may be one of methyl, ethyl, n-propyl, iso-propyl or cyclo-propyl. Combinations are also included in the invention. Preferably PAOx is ethyl.

Preferably, the polyoxazoline used in the method of the present invention is represented by the formula (la)

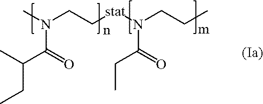

Thus, R₁ in Formula I is ethyl.

The molar ratio of PAOx to PsecBuOx (or ratio n/m) can be varied depending on the desired properties of the polyoxazoline. In particular, according to the invention the molar ratio PAOx/PsecBuOx is from 5/95 to 95/5. Preferably, the ratio is from 10/90 to 60/40, such as 20/80, 30/70 or 50/50. Variation of the ratio of PAOx and PsecBuOx allows for variation in polymer properties, in particular the LCST.

The molar ratio of PAOx to PsecBuOx can be determined by selecting the respective molar amounts of the starting materials for co-polymerization. It can also be determined in the final polymer by means of known analytical methods such as NMR.

The term “stat” should be understood as “statistical”, referring to statistical polymers, in this case being formed by one-pot statistical co-polymerization, e.g. of the monomer units PEtOx and PsecBuOx. Co-polymerization finds its particular use in the current invention in tuning the transition temperature of said co-polymers as variation of the ratio of both comonomers leads to a change in transition temperature. Incorporation of more PsecBuOx leads to a decrease in transition temperature.

The number average molecular weight of the polyoxazoline of the invention is preferably from 10 to 200 kDa (g/mol), more preferably from 30 to 100 kDa. The number average molecular weight also termed number average molar mass represents the average of the molecular masses of the individual macromolecules. It is determined by measuring the molecular mass of n polymer molecules, summing the masses and dividing it by n. The number average molecular mass of a polymer can be determined by various techniques such as but not limited to gel permeation chromatography, viscometry, vapor pressure osmometry, end-group determination or proton NMR.

Synthesis methods for these polymers are known and are for instance described in the above referenced patent applications and in WO2016008817.

The intended use of the present invention is tissue culture, more particularly three dimensional tissue culture. It is further intended in an embodiment that the cells are seeded on the template prior to embedding in the hydrogel and removing the template. This places several limitations on the materials:
first of all the critical temperature needs to be in a temperature range that is tolerated by the cells, meaning not too high and not too low. Generally mammalian cells are cultured at 37° C. and have very little tolerance for higher temperatures and some tolerance for brief periods in lower temperatures, such as 30° C. or 25° C.;
the time needed to solubilize the template must be sufficiently short, particularly with temperature in the lower tolerable range of the cells;
further, the material must be biocompatible, meaning it must be non-toxic and allow culturing of cells on or near the material. Importantly the material must be biocompatible both in the solid and dissolved state, as it is envisioned to seed cells on the template and then removing the template from the hydrogel by solubilizing it.

Polymers with a lower critical solution temperature have been described in the art, the inventors found that only be selecting a polyoxazoline polymer as described herein, the following issues are overcome:
the lower critical temperature is in arrange which is suitable for cell culture;
the polymer and particularly the template constructed from it is stable for a longer period of time when immersed in an aqueous solution above the T_{CP};
the template remains readily soluble when the aqueous solution is cooled to a temperature below the T_{CP}; and
the polymer is biocompatible allowing the seeding of cells on the template.

Although to some degree predictions can be made about the critical temperature when combining different co-polymers (see e.g. Lorson et al. (Biomaterials 178 pages 93-99)), the inventors found that other properties are not easily predicted. For example, when developing the present invention the inventors found that many combinations of co-polymers, which in theory should result in in a desirable critical temperature polymer and thus are suitable for the here intended use, were in fact not suitable for a variety of unexpected reasons.

A few examples are described in Reference examples 1 and 2. For example, the inventors found in some cases that although the critical temperature is in the desired range, the template does not remain stable long enough when exposed to water above the T_{CP} and loses its shape or even dissolves. In other cases the critical temperature of the template changes over time, resulting in a critical temperature too low to be tolerated by the cells. In yet other cases the critical temperature is either unexpectedly lower or needs to be sustained for a prolonged amount of time (too long a time of low temperature for the cells) before the template is rendered soluble. In yet other cases keeping the template at higher temperature in the solid state for a longer time resulted in rearrangement of the side chains and subsequent inability to dissolve. In yet other cases the template would not remain solid at temperatures tolerated by the cells (e.g. 37° C.).

None of these unexpected drawback could have been predicted from the prior art, more importantly however from the prior art it could not have been derived that the polymers describe herein overcome these, among other, issues and remain stable for a longer period of time at temperature ranges suitable for cells, are biocompatible, and can be dissolved at a temperature and time frame suitable for cells and remains biocompatible when dissolved.

### Structure Template

As described above, the polyoxazoline of the invention is used to create a template. The shape of the template is not particularly restricted and can be varied depending on the particular structure for cell growth that is created.

In an embodiment, the template is a fibre, cylinder or film. With fibre is meant an elongated body with a length dimension much greater than the transversal dimension, e.g. width and thickness. The term fibre includes a monofilament, a ribbon, a strip or a tape and the like, and can have a regular or an irregular cross-section. The fibre may have continuous lengths, known in the art as filaments.

With cylinder is meant an elongated body with a circular cross section having a length dimension greater than the diameter of the cross-section.

In an embodiment, the template comprises one or more microfilaments and the three dimensional structure obtained comprises one or more microchannels. These microfilaments typically have a diameter of from 0.5 to 5000 µm, preferably from 1 to 1000 µm, more preferably from 5 to 20 µm. The length of the microfilament is not particularly limited and is for example from 1 mm to 100 mm.

When the template comprises more than one filament, the filaments can be parallel or perpendicular. The filament can be a straight line, or it can be any shape suitable for the particular cells to be grown. Such shapes can be branched shapes or snake-like shapes. The filaments can have the same or different dimensions, creating for instance different diameters of the microchannels.

### Creating Template

The template can be created with known methods to deposit polymers such as additive manufacturing (3D printing). These methods include electrospinning, fused deposition modelling, thermoforming and casting.

A particular method of creating a microfiber template is melt electrospinning writing (MEW), see for instance Robinson TM, Hutmacher DW, Dalton PD. The Next Frontier in Melt Electrospinning : Taming the Jet. Adv Funct Mater. 2019;1904664.

Typical parameters for this method are a temperature of the polyoxazoline from 190 to 210° C., flow rate of 0.5 to 0.05 ml/hr, a voltage of +/- 2.5 kV to +/- 10 kV, a working distance of 5 mm to 20 mm, and a spinning speed of 8 to 100 mm/s.

A method of creating a filament template is fused deposition modelling (FDM), commonly referred to as 3D printing. This can be used alone to generate filament template structures or combined with MEW to create a multiscale template.

Typical parameters for this method are a temperature of the polyoxazoline from 190 to 220° C., applied extrusion pressure of 1 Bar to 5 Bar, and a deposition speed of 0 to 25 mm/s.

### Hydrogel

With hydrogel is meant a three dimensional hydrophilic network comprising hydrophilic gelators, in which water is the dispersion medium and that are capable of maintaining their structural integrity. Hydro-gels can be classified into polymeric hydrogels and low molecular weight hydrogels according to the composition of gelators. Polymeric hydrogels are preferred and the polymer scaffolds could be synthetic hydrophilic polymers or natural biomacromolecules.

As the hydrogel to embed the template, a known hydrogel can be used that is biocompatible and suitable as a scaffold in tissue engineering. Photocrosslinkable hydrogels can be used. These can be cross-linked by exposure to e.g. UV radiation. Hydrogels based on naturally occurring polymers can also be used. These can be cross-linked by temperature or by enzymatic processes.

Examples of these hydrogels are collagen, fibrin, poly(ethylene glycol) diacrylate (PEGDA), gelatin methacryloyl (GELMA), Matrigel®, alginate, agarose and polyacrylamide.

Step c) of the method of the invention, applying a hydrogel support onto the template to embed the template in the hydrogel, can be divided in two sub steps (i) applying an aqueous hydrogel pre-polymer; (ii) cross-linking the hydrogel pre-polymer.

In order to properly apply the hydrogel, a mould or support structure can be used that surrounds the template and that can be filled with the hydrogel pre-polymer. Concentrations of the aqueous hydrogel pre-polymer are those generally used for the type of hydrogel. Also the cross-linking conditions are the conditions known in the art. The cross-linking method and conditions should not interfere with the making, use and dissolution of the template. For instance, the hydrogel should not crosslink at a temperature that triggers polymer dissolution.

Although hydrogels exist that are prepared in other solvents than water, e.g. ethanol, followed by solvent exchange to water, aqueous solvents for the hydrogel preparation are preferred.

### Cells/Proteins

As described above the present invention provides three-dimensional structures for cell growth. The cells to be cultured can be added at different stages of the method.

According to an embodiment, the method further comprises a step of seeding cells on to the template before applying the hydrogel support.

The hydrophobicity of the polyoxazoline above the LCST makes it possible to adhere cells to the template structure prior to being embedded with the hydrogel. The cells are then allowed to attach to the hydrogel. After removal of the template, a microchannel structure lined with cells is obtained.

Since the removal of the dissolved template occurs via diffusion and, thus no flow is applied to potentially dislodge the cells from within the resulting channels, this sacrificial template approach represents an unique method to quickly form a cell-lined microchannel network with a three-dimensional hydrogel.

According to an embodiment, cells are added to the hydrogel before or after cross-linking.

Also combinations of these embodiments are possible, i.e. one type of cells is added to the hydrogel, and one type of cells is added to the polyoxazoline template. Further, if different microchannels are created, each microchannel can contain a different cell type.

It is also possible to have proteins or peptides coat the template surface prior to embedding within a hydrogel. These can facilitate cell adhesion and direct cell fate. If the template has been pre-coated with a protein, it then becomes possible to crosslink this protein to the hydrogel during the gelation process, thereby forming a microchannel network lined with a protein coating. Examples of proteins that can be used are collagens (many types) and laminins (many types) or growth factors (e.g. nerve growth factor (NGF), bone morphometric protein 2 (BMP-2)). Examples of peptides include RGD-based (Arg—Gly—Asp) peptide sequences and IKVAV-based (lle—Lys—Val—Ala—Val) peptide sequences.

A variety of cells can be grown with the structure created with the method of the invention by combining cells seeded on the template, cells mixed into the pre-gel solution to end up in the bulk hydrogel, as well as cells which can be introduced afterward to infiltrate the hydrogel through the resulting channel structures. A further possibility is to bioprint cells in a bulk hydrogel around the template, so that the cells in the bulk hydrogel have a useful localization, i.e. not ubiquitous. Examples cells that can be included as described include:
on template/channel wall: smooth muscle, glial cells, vascular endothelial cells, gut epithelial cells, endometrium epithelial cells, fallopian epithelial cells.
in gel: fibroblasts, macrophages, glial cells, stromal cells associated with respective epithelial tissue
in channel: neural cells (neurite extention - either axon or dendrite), vascular endothelial cells (directed cell migration and angiogenesis/sprouting through the microchannel structures).

### Dissolving Template

The step of removing the template comprises placing the template in an aqueous medium and decreasing the temperature of the template to below the LCST of the polyoxazoline polymer. This temperature is generally below 10° C., e.g. from 2 to 10° C.

The aqueous medium can be any water containing medium suitable for the purpose, in particular an aqueous medium compatible with cell culture. Examples include water, phosphate buffered saline (PBS) and a cell culture medium.

### Structure Obtained and Uses

The three-dimensional structure obtained with the method of the invention can be used for cell growth in vivo or in vitro. Examples are:
(in vitro) Complex tissue on a chip (e.g. complete vascularized and innervated full thickness epithelial tissue incorporating stromal cells and resident fibroblasts and macrophages)
(in vivo) Structured hydrogel filler for a nerve guide conduit comprised of an array of longitudinal microchannels within a biocompatible hydrogel in order to guide nerve/axon growth that also house Schwann (glial) cells well-distributed along the length of the microchannels to provide cell-cell adhesion and trophic support.
(in vivo) Generation of a large scale, prevascularized tissue for implantation, complete with a distributed, branched vascular network throughout and incorporating tracts for guided innervation and cell infiltration.
(in vitro) gut-on-a-chip applications
(in vitro) innervated-tissue-on-a-chip
(in vitro) vascular tissue-on-a-chip -> possible to look at, for example, cancer extravasation and metastic processes

When used here the term three-dimensional structure, when referring to the structure obtained by any of the method described herein refers to a hydrogel with embedded therein a template, or a hydrogel previously having a template embedded therein which was subsequently removed resulting in a cavity shaped like the template. The three-dimensional structure may comprise cells, but this is not mandatory as the cells may also be added later.

Therefore, in an embodiment the invention relates to use of a three-dimensional structure obtained with the method according to the invention for growing cells. The us may be the in vitro or ex vivo, or the use may be in vivo. Therefore in an embodiment the invention relates to the in vitro or ex vivo use of a three-dimensional structure obtained with the method according to the invention for growing cells. Alternatively the invention relates to an in vivo use. Therefore, in an alternative embodiment the invention relates to a three-dimensional structure obtained with the method according to the invention for use in the treatment of a subject in need thereof. In an embodiment the use comprises implanting the three-dimensional structure in a subject.

The in vitro, ex vivo or in vivo use may comprise one or more of the following:
culturing and/or proliferating cells in the three dimensional structure;
removing the template form the three dimensional structure;
embedding or seeding cells in the three dimensional structure.

### Device

According to another aspect, the invention provides a device for culturing cells comprising a polymer template embedded in a hydrogel, wherein the polymer is a polyoxazoline which is a copolymer of 2-ethyl-2-oxazoline and 2-sec-butyl-2-oxazoline. Further preferred embodiments of the polymer are as described above.

In particular, the device has a polymer template according to the invention comprising one or more microchannels having a diameter of from 0.5 to 5000 µm.

In an embodiment the device further comprises cells, preferably wherein the cells are seeded on or near the template.

### DRAWINGS

FIG. 1 illustrates an outline of the generic process for making the 3D structure and an example of use with cells.

FIG. 2 illustrates different layouts of cells that can be grown within a structure obtained according to the invention.

FIG. 3 illustrates a more complex layout of a structure obtained with the process of the invention.

FIG. 4 shows the melt viscosity of different PEtOx-stat-PsecBuOx variants.

FIG. 5 shows an example of neurite growth from an IPSC-derived neural cluster grown within a device. (A) a brightfield image showing the gel device with an access well containing two IPSC clusters (Scalebar: 2 mm). (B) A fluorescence image of the same region, with neurite growth visible (Scalebar: 2 mm). (C) a close up of region of axonal extension along a microchannel within the gel (Scalebar: 100 µm).

FIG. 6 shows the water contact angle at different temperatures for different polymers.

FIG. 7 shows an example of Schwann cells seeded onto a microfiber polymer template made from PEtOx-stat-PsecBuOx 20/80. Also shown is the resulting hydrogel after the template has been embedded, the collagen crosslinked, and the template dissolved after cooling to 4° C. for ~1 hr. The final 3D hydrogel culture device is comprised of microchannels lined with Schwann cells.

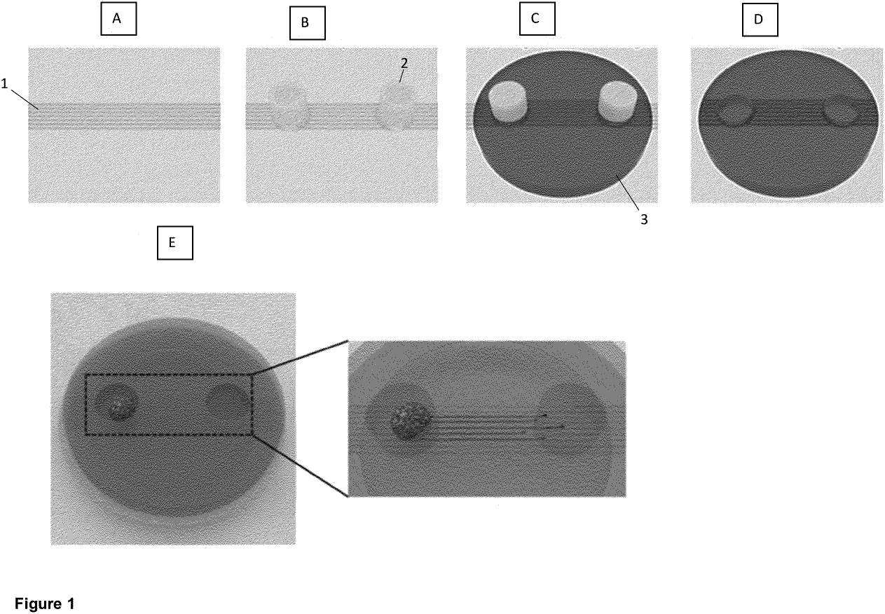

FIG. 1 illustrates an outline of the generic process flow for 3D hydrogel production. Starting from a template (1) of microfibers of 10 µm (FIG. 1A), larger sacrificial structures (2) are added to create additional features (in this case, 3 mm pillars) (FIG. 1B). The entire structure is embedded within a hydrogel (3) (FIG. 1C), the gel is crosslinked, and the template is dissolved (D). In this way, a 3D environment is created with small scale oriented microchannels to guide nerve growth, where the larger 3 mm posts form ‘access wells’ where cell bodies can be placed so that extending neurites can grow into said channels (E).

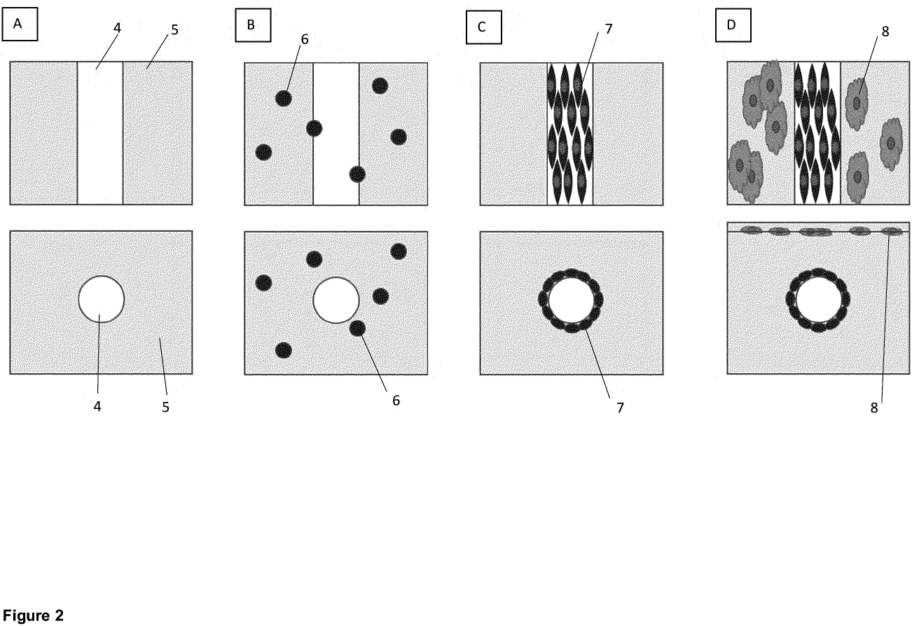

FIG. 2 illustrates possible configurations of the cells within the 3D structure. The top row shows the top view, the bottom row shows the cross-section. FIG. 2A illustrates formation of microchannels (4) in a hydrogel (5). FIG. 2B shows cells (6) embedded in the hydrogel (5). FIG. 2C shows cells (7) coated on the microchannel. FIG. 2D shows organized cell interaction between cells embedded in the hydrogel (8) and cells coated onto the microchannel (7).

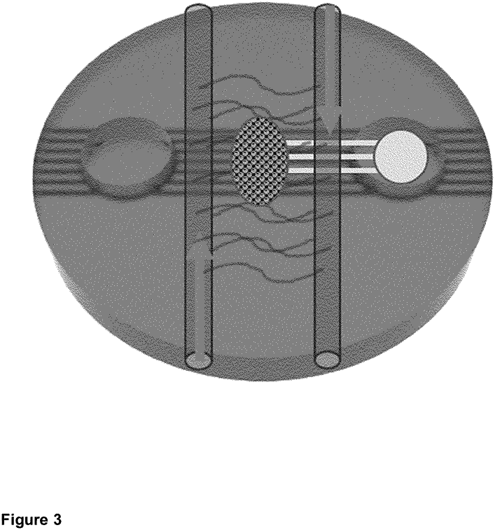

FIG. 3 shows a more complex form of the structure of FIG. 1. This is a platform with vascularization and innervation of tissue with large, perfusable channels connected to smaller cross-flow channels.

### EXAMPLES

To evaluate polymers for suitability in the invention, the polymers were submitted to thermally triggered dissolution. To perform this test, initially large filaments were extruded (approximately 1 mm in diameter) using the FDM method described above with a 150 µm diameter nozzle, a temperature of 200° C. and 5 Bar of applied pressure. The system used to extrude was a Bioinicia LE-100 Electrospinning system with custom MEW hardware consisting of a band heater controlled with a Temptron PID controller, which heats a metal syringe that is supported above the flat collector from the XY gantry system. These were placed within a Peltier heating/cooling element capable of maintaining liquid at temperatures ranging from 50° C. to approximately 4° C. This test allows to emulate the intended process flow where cells are seeded on the template at 37° C. And then the entire device is placed in a standard refrigerator (typically at 5° C.) to trigger template dissolution.

### Comparative Example PnPrOx

Polyn-propyl-2-oxazoline) with a molecular weight of 50 kg/mol was tested. This polymer has a LCST of about 30° C. Solubility was tested in a PBS solution. A filament was prepared via FDM as described above. Briefly, the polymer was heated to 200° C. within a metal syringe and extruded through a 150 µm diameter brass 3D printing nozzle with 5 Bar of air pressure. The filament was exposed to 37° C. for 10 minutes and then rapidly cooled to 5° C.

While the filament is maintained at 37° C., one can observe a change in filament opacity as it slowly absorbs some water but still maintains mechanical and morphological integrity. During the cooling process, one can observe the filament becoming rapidly more translucent as the material becomes increasingly more hydrophilic and, therefore, more soluble. However, it was observed that complete dissolution was only achieved by maintaining the filament at 5° C. for approximately 3 hrs.

In order to emulate a cell seeding process, whereby the template scaffold is seeded with cells prior to embedding and dissolution, we studied if the filament could be maintained at 37° C. for an extend period.

After maintaining the filament for 1 hr at 37° C., it was found that the filament no longer dissolved. This was ascribed to be a consequence of structural reorganization, i.e. of the semi-crystalline character of this polymer, resulting in partial crystallization. By maintaining the polymer at 37° C. (close to its Tg = 40° C.), the side changes were able to reorganize the fiber surface leading to hydrophobic fibres that can no longer dissolve.

A follow up experiment used smaller MEW generated fibres (approximately 20 µm) using a 150 µm diameter 3D printing nozzle, a temperature of 190° C., 0.25 Bar of pressure, -4 kV of applied voltage, 5 mm working distance, and a translation speed of 75 mm/s. Observing these fibres under similar dissolution conditions found that this phenomenon was consistent and not dependent on fibre/filament size or differences in surface-to-volume ratio (data not shown). For both sizes, samples were kept in the refrigerator for 3 days and the material still did not dissolve (data not shown).

### Further Comparative Examples

Further polyoxazoline variants were tested. The polymers had a number average molar mass above 30 kg/mol and were processed into fibers, using a 150 µm diameter 3D printing nozzle as described above for PnPrOx. The thermoresponsive dissolution behavior of the polymers was investigated in water with the following outcomes:
PcPrOx: Poly(2-c-Propyl-2-oxazoline); LCST ~30° C.
   Fast dissolution upon contact with water at 37° C., not suitable
PEtOx-stat-PnPrOx: Poly(2-ethyl-2-oxazoline)-stat-Poly(2-n-propyl-2-oxazoline; LCST 24-60° C.
   Fast dissolution upon contact with water when above the LCST, not suitable.
PEtOx-stat-PnBuOx: Poly(2-ethyl-2-oxazoline)-stat-Poly(2-n-butyl-2-oxazoline); LCST 20-30° C.:
   Fast dissolution upon contact with water above the LCST at 37° C. or 42° C., not suitable.
PsecBuOx: Poly(2-sec-Butyl-2-oxazoline) LCST ~5° C.
   No dissolution in water at 5° C., not suitable.
PEtOx-PnBuOx 70/30: Poly(2-ethyl-2-oxazoline)-stat-Poly(2-n-Butyl-2-oxazoline); LCST 22° C.
   Immediate dissolution upon contact with water, not suitable.
50:50 physical blend of Poly(2-ethyl-2-oxazoline) and PsecBuOx: Poly(2-sec-Butyl-2-oxazoline); LCST 5-60° C.
   Immediate dissolution upon contact with water, not suitable.

### Example 1 Poly(2-ethyl-2-oxazoline-stat-poly(2-sec-Butyl-2-oxazoline) (PEtOx-stat-PSecBuOx 30/70)

For this example, a copolymer of PEtOx and PSecBuOx was used with a molar ratio of 30/70 with an LCST of ~20° C. A filament was made of this copolymer as described above using a 150 µm diameter brass 3D printing nozzle connected to a metal syringe heated to 205° C. through which the molten polymer was extruded with 5 Bar of air pressure. Immersed in 37° C. PBS, the filament of this polymer maintained both its shape and mechanical properties. It also retained air bubbles on its surface, indicating hydrophobicity. This was maintained for 10 minutes, after which a rapid cooling phase showed the polymer beginning to change shape, losing the air bubbles on the surface, and then begin to dissolve starting at around 20° C. With the polymer maintained at 5° C., dissolution was complete after 5 minutes.

This dissolution assay shows that this polymer has the desired properties. A test of PEtOx-stat-PSecBuOx 30/70 kept at 37° C. overnight in PBS showed that the polymer still dissolved at 5° C.

Further fibres were manufactured with MEW with diameters from 15 to 20 µm. With a 150 µm diameter brass 3D printing nozzle, a deposition speed of 50 mm/s, a voltage of -7 kV, working distance of 10 mm, and a pressure of 1 Bar (100 kPa), temperatures were varied resulting in the following fibre diameters.

| Temp (°C) | Diameter (µm) |
|---|---|
| 193 | 14 ± 0.14 |
| 196 | 15 ± 0.1 |
| 200 | 18 ± 0.05 |

Further dissolution data were obtained for the polymers shown below in Table 1:

TABLE 1

| | Polymer PEtOx/PsecBuOx | Polymer Format | Preincubation Temp. | Preincubation Time | Swelling Temp. | Dissolution Temp. | Dissolution Time |
|---|---|---|---|---|---|---|---|
| 1 | 30:70 | Filament | 37° C. | 10 min | 19° C. | 5° C. | 18 min |
| 2 | 30:70 | Fibres | 37° C. | 10 min | 29° C. | 18° C. | 9 min |
| 3* | 30:70 | Fibres | 37° C. | 16 hrs | n/a | 4° C. | 30 min |
| 4* | 20:80 | Fibres | 21° C. | 10 min | n/a | 4° C. | ~1 hr |
| 5* | 20:80 | Fibres | 37° C. | 30 min | n/a | 4° C. | ~1 hr |
| 6* | 20:80 | Fibres | 37° C. | 2 hrs | n/a | 4° C. | ~1 hr |
| 7* | 10:90 | Fibres | 37° C. | 2 hrs | n/a | 4° C. | ~1 hr |

*These experiments were performed in an application setting, where the incubations and temperatures were applied as it would be during template use.

### Example 2 Rheology Data for PEtOx-stat-PsecBuOx 30/70;50/50;20/80

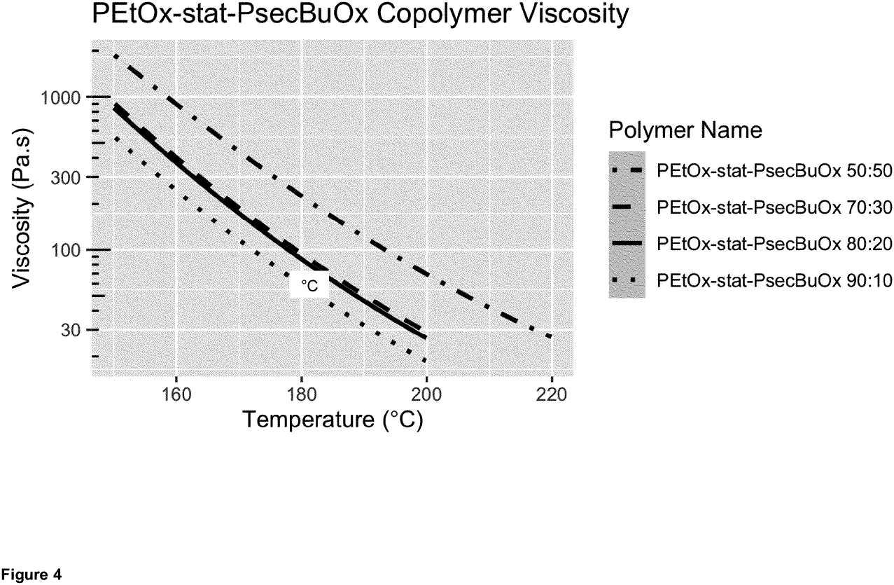

The viscosity of the polymer melt determines the flow rate for a pressure driven MEW system, were flows from 0.5 to 0.05 ml/hr are achieved by applying pressures from 0.5 to 1.5 Bar. For a more defined process parameter, melt viscosity was measured for the different PEtOx-stat-PsecBuOx variants with a parallel plate rheometer with heated plates. 100 mg of polymer was loaded between the plates, the temperature was raised to 200° C. to initially melt the polymer, and then the viscosity was determined by measuring the rotational force required to subject the polymer melt to a cyclic of 1° angular displacement. The sample was then cooled slowly by 1° C./min. This data shows that, for the same typical range of operating temperatures (from 190 to 200° C.) the PEtOx-stat-PsecBuOx 30:70 and PEtOx-stat-PsecBuOx 20:80 achieve approximately similar melt viscosity. For the PEtOx-stat-PsecBuOx 50:50, a much higher viscosity is measured for the same range, indicating that lower flow rates will be generated and that higher temperatures (210-220° C.) are required for this polymer to be processed in a similar manner. The results are shown in FIG. 4.

### Example 3 Formation Microchannels and Cell Growth

In order to test the polymer of Example 1 (PEtOx-stat-PSecBuOx 30/70) embedded in a hydrogel, a polymer mould was manufactured having polycaprolactone (PCL) pillars on a frame (poly methyl methacrylate PMMA).

Aligned fibres of the polymer were produced with MEW having a diameter of 15 µm.

The pillars were placed on top of aligned fibres and, taking advantage of the relatively low melt temperature of PCL (Tm=~65° C.) compared to polyoxazolines, this assembly was placed on a hotplate held between 65° and 80° C. The bottom of the pillars were therefore exposed to a temperature above the PCL Tm and began to soften and flow, merging with the underlying fibres. After 2 to 10 seconds, the assembly was removed from the hotplate and allowed to cool. The result was an array of aligned fibres suspended between PCL pillars and, with the help of the PMMA frame, this was suspended into a hydrogel mould.

An example hydrogel that has been successfully used is rat tail collagen type 1, at a final concentration of 4 mg/ml in PBS. This pre-gel solution is typically maintained on ice (~0° C.) to maintain a liquid solution. Once this solution is heated to 37° C., a self-assembly of collagen proteins is triggered and causes the solution to form a stable, irreversible gel. Crosslinking begins to notably occur around 20° C., albeit slowly.

For this application, the collagen pre-gel solution is pre-warmed to 15° C., giving a pot-life of approximately 15 minutes before collagen crosslinking becomes too viscous to be pipetted but warm enough to limit template dissolution upon hydrogel application. This solution is then applied to the template and everything is quickly warmed to 37° C. to finalize crosslinking.

In general, it’s better to apply the hydrogel pre-polymer solution above the LCST of the template polymer, when possible. When not possible, then having the temperature as close to the LCST is a workable compromise.

Once the gel was applied and crosslinked, the device was cooled to 5° C. for approximately 15 to 30 minutes to complete dissolution. Once the fibres were dissolved, the PCL was no longer bound to the gel and the PCL+PMMA frame was removed, leaving behind a hydrogel with defined access wells connected by microchannels.

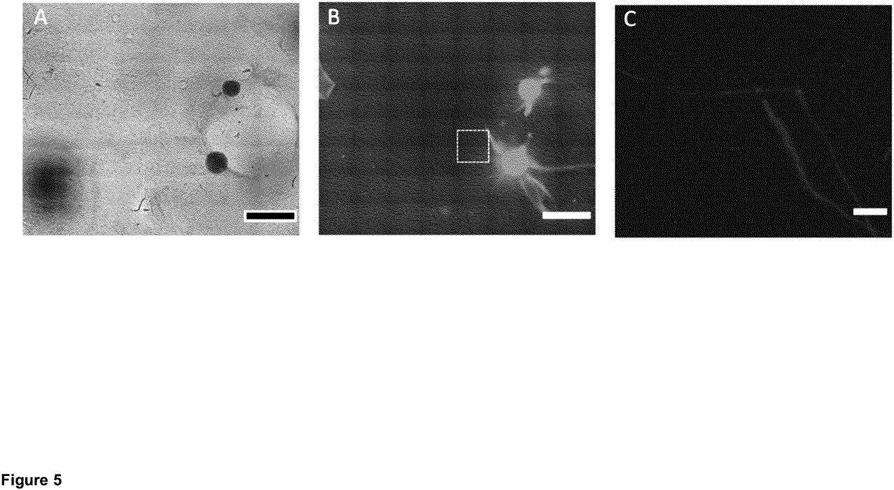

Next, IPSC-derived human neural cells were placed in the resulting access well. The outgrowth of neurons through the resulting channels was recorded and is shown in FIG. 5.

### Example 4 Determination of Hydrophobicity

Water contact angle was measured to determine the hydrophobicity of the polymer at different temperatures. This confirms the LCST behaviour and also allows one to estimate the ability of cells to adhere to the polymer surface, since it is widely recognized that cells prefer a moderately hydrophobic surface (40° to 60° WCA). This was measured by spin coating a thin film of the polymer (dissolved in chloroform) onto a glass coverslip and then placing this coating coverslip onto a flat Peltier element. This was incorporated into a WCA measurement system along with a heated syringe of PBS which maintained PBS solution at approximately 60° C. A drop of this warmed PBS was deposited onto the polymer film and the WCA was monitored via time lapse over a 2 minute period until the angle had stabilize. The angle was automatically according to the proprietary software of the WCA system. The WCA of the last 30 seconds was observed to be stable and, therefore, averaged to produce the ‘final’ WCA. This was measured for a number of temperatures ranging from 37° C. to 5° C., reflecting the transition from cell incubator to refrigerator, respectively.

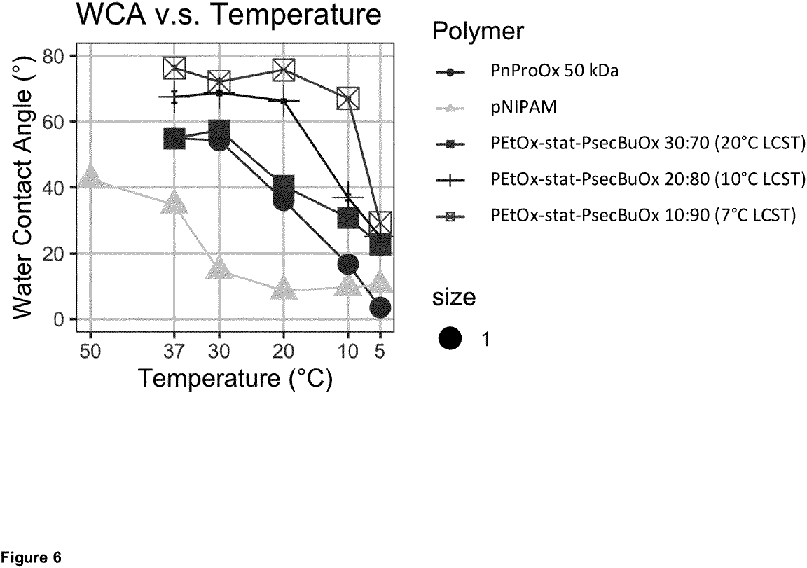

Data for the polymer of Example 1 (PEtOx-stat-PSecBuOx 30/70) were compared with state of the art polymer poly(N-isopropylacrylamide) (pNIPAM) and a different poly-oxazoline not according to the invention: poly(2-n-propyl-2-oxazoline, (PnProOx) with a Mw of 50 kDa. The results are shown in FIG. 6.

### Example 5 Cell Seed Efficacy

Further experiments were done to evaluate cell seeding efficiency. After preparing a solution of PEtOx-stat-PSecBuOx 30/70 in water, this was added to a tissue culture well plate and the water was allowed to evaporate, forming a thin film on the bottom of each well. Primary rat Schwann cells were seeded in each well and allowed to adhere and grow over a 3 day period. Cells appeared to adhere well, though the cell morphology was not comparable to normal tissue culture plastic.

After seeding and maintenance for 3 days, the well plate was cooled to 4° C. for 15 minutes to allow the polymer to dissolve. The culture medium was collected and spun down to collect the cells in the bottom of a 15 ml tube. Cells were carefully collected, resuspended in clean medium and replaced in a fresh wellplate. They were observed to adhere again, indicating that they had survived the process and remained viable.

### Example 6 Polymer Synthesis

### Monomer Synthesis and Purification

2-Ethyloxazoline (EtOx; Polymer Chemistry Innovations) was purified via fractional distillation and purification over barium oxide. 2-sec-butyloxazoline (secBuOx) was synthesized via the Witte-Seeliger method (Witte et al., Ann. Chem. 1974), from their corresponding nitrile, i.e. 2-methylbutyronitrile. The purification of secBuOx was carried out similarly to that of EtOx.

### Initiator

Trifluoromethanesulfonic acid was purchased from Sigma Aldrich and used as received.

### Polymerization

Polymers were synthesized with a target number of repeating units typically from 300 to 500. A typical polymer synthesis involves the administration of secBuOx monomer and a comonomer, such as EtOx, in a microwave reaction vial under an inert atmosphere. Both monomers are dosed in the desired molar ratio. Subsequently, the initiator is added in the required quantity to match the desired polymer length.

The vial is sealed under an inert atmosphere and placed in a microwave reactor (Biotage Initiator) at a temperature of 120° C. for 60 minutes.

### Representative Example of a Polymerization: Poly[(2-ethyl-2-oxazoline)₁₂₀-stat-(2-sec-butyl-2-oxazoline)₂₈₀ (PEtOx₁₂₀-stat-PsecBuOx₂₈₀)

An oven dried 20 mL microwave reactor vial is transferred to a glovebox (Vigor technologies) with a water content below 0.1 ppm. The vial is loaded with a stirring bar, 3.060 mL of EtOx (3.005 g, 30.3 mmol) and 9.67 mL of sec-BuOx (8.99 g, 70.7 mmol). The vial is closed and transferred out of the glovebox. A 25 mL Schlenk flask is dried, fitted with a septum, connected to a Schlenk line and filled with Argon. 10 mL of dry acetonitrile are injected into the flask, followed by 0.800 mL of trifluoromethanesulfonic acid. This stock solution is homogenized, and 0.279 mL initiator (0.038 g., 0.25 mmol) are taken with a syringe. The solution is then injected into the microwave vial containing the monomer mixture.

The vial is placed in the microwave synthesizer and heated to 120° C. for 60 minutes.

### Purification and Characterization

The synthesized polymers were dissolved in dichloromethane and purified by washing three times with a saturated solution of NaHCO₃ and once with water.

The polymers where characterized by 1H-NMR spectroscopy and size exclusion chromatography (SEC) on an Agilent 1260-series HPLC system equipped with a 1260 online degasser, a 1260 ISO-pump, a 1260 automatic liquid sampler (ALS), a thermo-stated column compartment (TCC) at 50° C. equipped with two PLgel 5 µm mixed-D columns in series, a 1260 diode array detector (DAD) and a 1260 refractive index detector (RID). The used eluent is N,N-dimethylacetamide (DMA) containing 50 mM of lithium chloride at an optimized flow rate of 0.5 mL/min. The spectra were analyzed using the Agilent ChemStation software with the GPC add on. Molar mass (Mn and Mp) and dispersity (Ð) values were calculated against polymethylmethacrylate molar mass standards from PSS.

The characterization data for the synthesized polymers is summarized in Table 2.

TABLE 2

Overview size-exclusion chromatography data for the PAOx copolymers

| Batch | Mₚ (PMMA) | Mₙ (PMMA) | Ð | T_{CP} |
|---|---|---|---|---|
| | kDa | kDa | - | °C |
| PEtOx₈₀-stat- PsecBuOx₃₂₀¹ | 49,200 | 28,100 | 2.04 | 13 |
| PEtOx₁₂₀-stat- PsecBuOx₂₈₀² | 53,000 | 38,600 | 1.97 | 22 |
| PEtOx₂₀₀-stat- PsecBuOx₂₀₀³ | 101,900 | 43,200 | 2.16 | 30 |
| PsecButOx₃₀₀ | 54,000 | 63,000 | 1.14 | < 4 |

¹ Also referred to as PEtOx-stat-PSecBuOx 30/70
² Also referred to as PEtOx-stat-PSecBuOx 20/80
³ Also referred to as PEtOx-stat-PSecBuOx 50/50

### Example 7 Fiber Template Cell Seeding and Cell-Laden Channel Formation

Experiments were performed to evaluate cell seeding onto a microfiber template and subsequent embedding and dissolution of this cell-laden fiber into a collagen hydrogel to form cell-lined microchannels within the gel. After preparing microfibers of PEtOx-stat-PSecBuOx 20/80 via melt elecrowriting, this was placed in a tissue culture well plate and a cell suspension of primary rat Schwann cells in cell culture medium with 10 wt% dextran was added to the well and allowed to adhere for a 2 hr period. Cells appeared to adhere well.

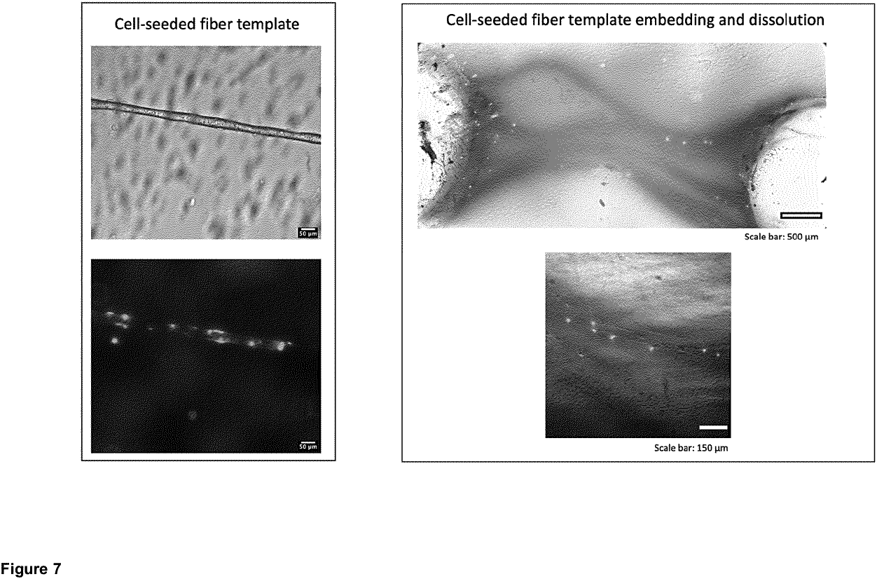

The cell-laden microfiber templates were embedded in a 4 mg/ml collagen pre-gel solution at 15° C., after which the resulting device was warmed to 37° C. and held at that temperature for 30 minutes to form a crosslinked collagen hydrogel. This was then cooled to 4° C. by placing the device in a fridge for approximately 1 hr, during which time the polymer template microfibers dissolved. The resulting device consistent of a hydrogel with a 3D microchannel network with cells incorporated within the microchannels. The results are shown in FIG. 7.

1. A method for creating a three-dimensional structure for cell growth comprising the steps of:
a) providing a polymer;
b) creating a template from the polymer;
c) applying a hydrogel support onto the template to embed the template in the hydrogel;
d) removing the template;
wherein the polymer is a polyoxazoline which is a PsecBuOx-stat-PAOx co-polymer represented by formula (I); 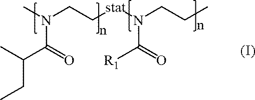
wherein
each R₁ is independently selected from -methyl, -ethyl, -n-propyl, cyclo-propyl and isopropyl;
the sum of m and n is from 20 to 1000, preferably from 200 to 700, most preferably from 300 to 500.

2. The method according to claim 1, wherein R₁ is ethyl.

3. The method according to claim 1, wherein the polyoxazoline has a number average molecular weight of from 10 to 200 kDa.

4. The method according to claim 1, wherein the template is a fibre, cylinder or film.

5. The method according to claim 4, wherein the template comprises one or more microfilaments and the three dimensional structure obtained comprises one or more microchannels.

6. The method according to claim 5, wherein the one or more microfilaments have a diameter from 0.5 to 5000 µm, preferably from 1 to 1000 µm, more preferably from 5 to 20 µm.

7. The method according to claim 1, wherein step (c) comprises:
(i) applying an aqueous hydrogel pre-polymer on the template
(ii) cross-linking the hydrogel prepolymer.

8. The method according to claim 1, wherein the method further comprises a step of seeding cells on to the template before applying the hydrogel support.

9. The method according to claim 1, wherein the step of removing the template comprises placing the template in an aqueous medium and decreasing the temperature of the template to below the LCST of the polyoxazoline polymer.

10. The method according to claim 9, wherein the temperature is decreased to below 10° C.

11. The method according to claim 1, wherein step b) comprises depositing the polyoxazoline polymer by electrospinning, fused deposition modelling, thermoforming or casting.

12. The method according to claim 1, wherein the cells are selected from the group consisting of smooth muscle cells, glial cells, vascular endothelial cells, gut epithelial cells, endometrium epithelial cells, fallopian epithelial cells, fibroblasts, macrophages, glial cells, stromal cells associated with respective epithelial tissue, neural and vascular endothelial cells.

13. Use of a three-dimensional structure obtained with the method of claim 1 for growing cells in vivo or in vitro.

14. Device for culturing cells comprising a polymer template embedded in a hydrogel, wherein the polymer is a PsecBuOx-stat-PAOx co-polymer represented by formula (I); 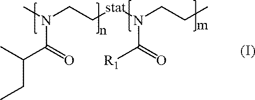
wherein
each R₁ is independently selected from -methyl, -ethyl, -n-propyl, cyclo-propyl and isopropyl;
preferably R₁ is ethyl;
the sum of m and n is from 20 to 1000, preferably from 200 to 700, most preferably from 300 to 500.

15. Device according to claim 14, wherein the polymer template comprises one or more microchannels having a diameter of from 0.5 to 5000 µm.
